(19) United States
(12) Patent Application Publication
(10) Pub. No.: US 2014/0227282 A1
(43) Pub. Date: Aug. 14, 2014

(54) THERAPEUTIC AGENT FOR PANCREATIC CANCER AND/OR BILIARY TRACT CANCER

(71) Applicants: AJINOMOTO CO., INC., Tokyo (JP); NATIONAL UNIVERSITY CORPORATION KOCHI UNIVERSITY, Kochi-shi (JP)

(72) Inventors: Shinobu NISHITANI, Kawasaki-shi (JP); Kazuhiro HANAZAKI, Kochi-shi (JP); Toshiji SAIBARA, Kochi-shi (JP)

(73) Assignees: AJINOMOTO CO., INC., Tokyo (JP); NATIONAL UNIVERSITY CORPORATION KOCHI UNIVERSITY, Kochi-shi (JP)

(21) Appl. No.: 14/255,411

(22) Filed: Apr. 17, 2014

Related U.S. Application Data
(63) Continuation of application No. PCT/JP2012/076879, filed on Oct. 18, 2012.

(30) Foreign Application Priority Data
Oct. 18, 2011 (JP) 2011-229116

(51) Int. Cl. A61K 31/7068; A61K 31/198; A61K 31/513

(52) U.S. Cl. CPC A61K 31/7068; A61K 31/513; A61K 31/198
USPC 424/141.1; 514/49; 424/649

(57) ABSTRACT

The problem of the present invention is to provide a highly effective therapeutic drug for pancreatic cancer and/or biliary tract cancer.

A therapeutic drug for pancreatic cancer and/or biliary tract cancer containing the following (1) and (2) as essential components:
(1) at least one kind of branched-chain amino acid selected from the group consisting of isoleucine, leucine and valine,
(2) gemcitabine or a salt thereof.

### CROSS REFERENCES TO RELATED APPLICATIONS

This application is a continuation of International Patent Application No. PCT/JP2012/076879, filed on Oct. 18, 2012, and claims priority to Japanese Patent Application No. 2011-229116, filed on Oct. 18, 2011, all of which are incorporated herein by reference in their entireties.

### BACKGROUND OF THE INVENTION

1. Field of the Invention

The present invention relates to a therapeutic drug for pancreatic cancer and/or biliary tract cancer, comprising a branched-chain amino acid and gemcitabine or a salt thereof as essential components.

2. Background of the Invention

In our country, about 21,000 people annually die of pancreatic cancer developed in the pancreas surrounded by stomach, duodenum, small intestine, large intestine, liver, gall bladder, spleen and the like, which is 2.2-fold compared to 20 years ago and is rapidly increasing. Since pancreatic cancer often does not show characteristic clinical symptoms in initial stages, early detection is not easy. Therefore, patients diagnosed with pancreatic cancer generally show poor prognosis. The average survival period after diagnosis is 3-5 months and the 5 year survival rate is only about 15%.

The primary choice of the treatment of pancreatic cancer is surgical resection. However, since the disease has, in many cases, already progressed and spread when found, surgery is possible only in a relatively small number of cases. The primary choice for chemotherapy of inoperable pancreatic cancer is gemcitabine hydrochloride, which is an antimetabolite. However, the treatment effect thereof is not higher than in other solid tumors. Under the circumstances, a highly effective treatment method is desired.

In general cancer chemotherapy, a multidrug therapy using two or more agents with different action mechanisms in combination is employed as a method for enhancing the effectiveness of the anti-cancer agents. In chemotherapy of pancreatic cancer, a combination therapy of gemcitabine and various medicaments is performed and, for example, combined use of gemcitabine and NSAIDs (patent document 1), combined use of gemcitabine and EGFR kinase inhibitor (patent document 2), combined use of gemcitabine and erlotinib (patent document 3), combined use of gemcitabine and endostatin (non-patent document 1), combined use of gemcitabine and 5-fluorouracil (non-patent document 2), combined use of gemcitabine and ascorbic acid (non-patent document 3) and the like have been reported. However, none of these treatment methods can improve the survival rate remarkably, and the combined use often enhances the side effects. Therefore, a combined use treatment method of medicaments that limit side effects while affording effectiveness is urgently demanded for the treatment of pancreatic cancer.

LIVACT (registered trade mark) is a preparation composed of three kinds of branched-chain amino acids of isoleucine, leucine and valine (BCAA). It is a medicament developed to correct the Fisher ratio, increase the serum albumin concentration, and improve hypoalbuminemia, by oral supplementation of BCAA at an appropriate ratio. Non-patent document 4 reports that a decrease in the serum albumin concentration within one month after surgery is one of the factors that worsen prognosis in the treatment of pancreatic cancer. However, it has not been reported heretofore that BCAA affords an action to potentiate an anticancer activity of gemcitabine on pancreatic cancer and biliary tract cancer.

### DOCUMENT LIST

### Patent Documents

patent document 1: JP-A-2003-514017
patent document 2: JP-A-2008-501652
patent document 3: JP-A-2011-506492

### Non-Patent Documents

non-patent document 1: Biomedicine & Pharmacotherapy 64 (2010) 309-312
non-patent document 2: Cancer 117 (2011) 2620-2628
non-patent document 3: Free Radical Biology & Medicine 50 (2011) 1610-1619
non-patent document 4: World J Surg 33 (2009) 104-110

### SUMMARY OF THE INVENTION

### Problems to be Solved by the Invention

The problem to be solved by the present invention is to provide a highly effective therapeutic drug for pancreatic cancer and/or biliary tract cancer.

### Means of Solving the Problems

The present inventors have conducted intensive studies in an attempt to solve the aforementioned problem and surprisingly found that combined use of at least one kind of branched-chain amino acid selected from the group consisting of isoleucine, leucine and valine, and gemcitabine or a salt thereof enhances an anticancer activity thereof on pancreatic cancer and biliary tract cancer, and conducted further studies based on such finding, which resulted in the completion of the present invention.

Accordingly, the present invention provides the following.

[1] A therapeutic drug for pancreatic cancer and/or biliary tract cancer, comprising the following (1) and (2) as essential components

(1) at least one kind of branched-chain amino acid selected from the group consisting of isoleucine, leucine and valine, and

(2) gemcitabine or a salt thereof.

[2] The therapeutic drug for pancreatic cancer and/or biliary tract cancer of the above-mentioned [1], further comprising the following component (3)

(3) at least one kind of compound selected from the group consisting of a 5-fluorouracil compound, a platinum compound, a taxane compound, a vinca alkaloid compound, an anticancer tyrosine kinase inhibitory compound, and an anticancer monoclonal antibody.

[3] The therapeutic drug for pancreatic cancer and/or biliary tract cancer of the above-mentioned [1], comprising a preparation containing component (1) in combination with a preparation containing component (2).
[4] The therapeutic drug for pancreatic cancer and/or biliary tract cancer of the above-mentioned [3], further comprising a preparation containing component (3) in combination.
[5] The therapeutic drug for pancreatic cancer and/or biliary tract cancer of the above-mentioned [2] or [4], wherein the component (3) is a 5-fluorouracil compound.
[6] The therapeutic drug for pancreatic cancer and/or biliary tract cancer of the above-mentioned [5], wherein the 5-fluorouracil compound is at least one kind of compound selected from the group consisting of 5-fluorouracil, tegafur, tegafur-gimeracil-oteracil potassium, and capecitabine.
[7] The therapeutic drug for pancreatic cancer and/or biliary tract cancer of any of the above-mentioned [1]-[6], wherein the component (1) is composed of three kinds of branched-chain amino acids of isoleucine, leucine and valine.
[8] The therapeutic drug for pancreatic cancer and/or biliary tract cancer of the above-mentioned [7], wherein the weight ratio of isoleucine, leucine, and valine is 1:1-3:0.5-2.0.
[9] The therapeutic drug for pancreatic cancer and/or biliary tract cancer of the above-mentioned [7] or [8], wherein the weight ratio of isoleucine, leucine, and valine is 1:1.5-2.5:0.8-1.7.
[10] The therapeutic drug for pancreatic cancer and/or biliary tract cancer of any of the above-mentioned [7]-[9], wherein the weight ratio of isoleucine, leucine, and valine is 1:1.9-2.2:1.1-1.3.
[11] The therapeutic drug for pancreatic cancer and/or biliary tract cancer of any of the above-mentioned [1]-[10], wherein the component (2) is gemcitabine hydrochloride.
[12] The therapeutic drug of any of the above-mentioned [1]-[11], wherein the pancreatic cancer and/or biliary tract cancer is advanced pancreatic cancer.
[13] An enhancer of an anticancer activity of gemcitabine or a salt thereof against pancreatic cancer and/or biliary tract cancer, comprising at least one kind of branched-chain amino acid selected from the group consisting of isoleucine, leucine and valine.
[14] The agent of the above-mentioned [13], comprising three kinds of branched-chain amino acids of isoleucine, leucine and valine.
[15] A method of treating pancreatic cancer and/or biliary tract cancer, comprising administering an effective amount of the following components (1) and (2) to a patient

(1) at least one kind of branched-chain amino acid selected from the group consisting of isoleucine, leucine and valine, and

(2) gemcitabine or a salt thereof.

[16] The treatment method of the above-mentioned [15], further comprising administering an effective amount of the following component (3)

(3) at least one kind of compound selected from the group consisting of a 5-fluorouracil compound, a platinum compound, a taxane compound, a vinca alkaloid compound, an anticancer tyrosine kinase inhibitory compound, and an anticancer monoclonal antibody.

[17] Use of the following components (1) and (2) for the treatment of pancreatic cancer and/or biliary tract cancer

(1) at least one kind of branched-chain amino acid selected from the group consisting of isoleucine, leucine and valine, and

(2) gemcitabine or a salt thereof.

[18] Use of the above-mentioned [17], further comprising use of the following component (3) in combination

(3) at least one kind of compound selected from the group consisting of a 5-fluorouracil compound, a platinum compound, a taxane compound, a vinca alkaloid compound, an anticancer tyrosine kinase inhibitory compound, and an anticancer monoclonal antibody.

### Effect of the Invention

According to the present invention, a highly effective therapeutic drug for pancreatic cancer and/or biliary tract cancer (including bile duct cancer, gall bladder cancer, papillary cancer) can be provided.

According to the present invention, moreover, an enhancer of an anticancer activity of gemcitabine or a salt thereof against pancreatic cancer and/or biliary tract cancer (including bile duct cancer, gall bladder cancer, papillary cancer) can be provided.

### BRIEF DESCRIPTION OF THE DRAWINGS

FIG. 1 is a graph showing the number of surviving cells of Panc-1 cells after culture in media 1-1 to 1-3 for 72 hr.

FIG. 2 is a graph showing the number of surviving cells of Panc-1 cells after culture in media 2-1 to 2-3 for 72 hr.

FIG. 3 is a graph showing the volume of tumor.

FIG. 4 is a graph showing the weight of tumor.

### DESCRIPTION OF EMBODIMENTS

The therapeutic drug for pancreatic cancer and/or biliary tract cancer (including bile duct cancer, gall bladder cancer, papillary cancer) of the present invention comprises

(1) at least one kind of branched-chain amino acid selected from the group consisting of isoleucine, leucine and valine (component (1)), and
(2) gemcitabine or a salt thereof ((component (2)) as essential components.

### [Component (1)]

The component (1) of the therapeutic drug for pancreatic cancer and/or biliary tract cancer of the present invention is any one or more kinds of branched-chain amino acids of isoleucine, leucine and valine, and preferably composed of three kinds of branched-chain amino acids of isoleucine, leucine and valine.

As isoleucine, leucine and valine, any of an L-form, a D-form and a DL-form can be respectively used. Preferred are an L-form and a DL-form, and more preferred is an L-form.

Isoleucine, leucine and valine can be used not only in a free form but also in the form of a salt. While the form of a salt is not particularly limited as long as it is a pharmaceutically acceptable salt of isoleucine, leucine or valine, for example, acid addition salt, salt with a base and the like can be mentioned.

Examples of the acid for forming a pharmaceutically acceptable salt of isoleucine, leucine or valine include inorganic acids such as hydrogen chloride, hydrogen bromide, sulfuric acid, phosphoric acid and the like; organic acids such as acetic acid, lactic acid, citric acid, tartaric acid, maleic acid, fumaric acid, monomethyl sulfuric acid and the like, and the like.

Examples of the base for forming a pharmaceutically acceptable salt of isoleucine, leucine or valine include inorganic bases such as sodium, potassium, calcium, ammonia and the like; organic bases such as ethylenediamine, propylenediamine, ethanolamine, monoalkyl ethanolamine, dialkyl ethanolamine, diethanolamine, triethanolamine and the like, and the like.

The salt of isoleucine, leucine or valine may be a hydrate (hydrate salt). Examples of such hydrate include 1-6 hydrates and the like.

When the component (1) is composed of three kinds of branched-chain amino acids of isoleucine, leucine and valine, the weight ratio of isoleucine, leucine and valine is generally 1:1-3:0.5-2.0, preferably 1:1.5-2.5:0.8-1.7, particularly preferably 1:1.9-2.2:1.1-1.3.

In the present invention, the “weight ratio” means a ratio of the weight of each component in the preparation. For example, when isoleucine, leucine and valine are contained in one preparation, it means a ratio of individual contents. Alternatively, when each of them is contained in plural preparations singly or in any combination, it means a ratio of the total amount of each component contained in respective preparations.

### [Component (2)]

Gemcitabine to be used as component (2) in the therapeutic drug for pancreatic cancer and/or biliary tract cancer of the present invention is (+)-2′-deoxy-2′,2′-difluorocytidine gemcitabine (CAS 95058-81-4).

While the salt of gemcitabine is not particularly limited as long as it is a pharmaceutically acceptable salt, for example, a salt with an acid, a salt with a base and the like can be mentioned.

Examples of the acid for forming a pharmaceutically acceptable salt of gemcitabine include inorganic acids such as hydrochloric acid, hydrogen bromide, sulfuric acid, phosphoric acid and the like; organic acids such as formic acid, acetic acid, lactic acid, succinic acid, citric acid, tartaric acid, maleic acid, fumaric acid, stearic acid, benzoic acid, methanesulfonic acid, benzenesulfonic acid, p-toluenesulfonic acid, trifluoroacetic acid, monomethyl sulfuric acid and the like, and the like.

Examples of the base for forming a pharmaceutically acceptable salt of gemcitabine include inorganic bases such as sodium, potassium, calcium, magnesium, ammonia and the like; organic bases such as trimethylamine, triethylamine, pyridine, picoline, dicyclohexylamine, N,N′-dibenzylethyleneamine, arginine, lysine and the like, and the like.

Gemcitabine or a salt thereof may be crystalline or amorphous. When crystalline polymorphism is present, it may be a single crystal form of any of them or a mixture thereof.

Component (2) is preferably gemcitabine hydrochloride.

Gemcitabine or a salt thereof can be produced by a known method. In addition, gemcitabine hydrochloride can also be obtained by purchasing Gemzar (registered trade mark) from Eli Lilly & Co. and the like.

The therapeutic drug for pancreatic cancer and/or biliary tract cancer of the present invention may contain other anti-cancer agent (component (3)) in addition to the aforementioned components (1) and (2). When other anti-cancer agent is contained, a higher anticancer activity can be obtained.

### [Component (3)]

Other anti-cancer agent used as component (3) in the therapeutic drug for pancreatic cancer and/or biliary tract cancer of the present invention is not particularly limited as long as it can be used in combination with gemcitabine or a salt thereof. For example, a 5-fluorouracil compound, a platinum compound, a taxane compound, a vinca alkaloid compound, an anticancer tyrosine kinase inhibitory compound, an anticancer monoclonal antibody and the like can be mentioned. Preferred is a 5-fluorouracil compound.

Examples of the 5-fluorouracil compound include 5-fluorouracil, tegafur, tegafur-gimeracil-oteracil potassium, capecitabine and the like.

Examples of the platinum compound include cisplatin, carboplatin and the like.

Examples of the taxane compound include docetaxel, paclitaxel and the like.

Examples of the vinca alkaloid compound include vinblastine, vincristine and the like.

Examples of the anticancer tyrosine kinase inhibitory compound include gefitinib, erlotinib, sorafenib and the like.

Examples, of the anticancer monoclonal antibody include rituximab, trastuzumab and the like.

All of these are commercially available.

### [Preparation]

The therapeutic drug for pancreatic cancer and/or biliary tract cancer of the present invention can be formulated as a preparation by mixing components (1) and (2), and component (3) as necessary with a pharmacologically acceptable carrier according to a method known per se. The obtained preparation can be administered orally or parenterally (e.g., topical, rectal, intravenous administration etc.).

Preferable specific examples of the preparation of the therapeutic drug for pancreatic cancer and/or biliary tract cancer of the present invention include

a preparation containing a branched-chain amino acid composed of isoleucine, leucine and valine, and gemcitabine hydrochloride;

a preparation containing a branched-chain amino acid composed of isoleucine, leucine and valine, gemcitabine hydrochloride, and 5-fluorouracil; and

a preparation containing a branched-chain amino acid composed of isoleucine, leucine and valine, gemcitabine hydrochloride, and tegafur-gimeracil-oteracil potassium.

The preparation of the therapeutic drug for pancreatic cancer and/or biliary tract cancer of the present invention may be one for oral administration or parenteral administration and, for example, injection (intramuscular injection, intravenous injection), liquids such as tubal liquid and the like, powder, fine granule, granule, tablet, capsule, cream, suppository and the like can be mentioned.

Examples of the pharmacologically acceptable carrier include lactose, glucose, D-mannitol, starch, crystalline cellulose, calcium carbonate, kaolin, starch, gelatin, hydroxypropyl cellulose, hydroxypropyl methylcellulose, polyvinylpyrrolidone, ethanol, carboxymethylcellulose, carboxymethylcellulose calcium salt, magnesium stearate, talc, acetylcellulose, titanium oxide, benzoic acid, p-hydroxybenzoate ester, sodium dehydroacetate, gum arabic, tragacanth, methylcellulose, egg-yolk, surfactant, sucrose, simple syrup, citric acid, distilled water, glycerol, propylene glycol, macrogol, disodium hydrogen phosphate, sodium dihydrogen phosphate, sodium phosphate, sodium chloride, phenol, thimerosal, sodium bisulfite and the like.

### [Dose, Administration Method]

In the therapeutic drug for pancreatic cancer and/or biliary tract cancer of the present invention, the dose of component (1) varies depending on the pathology and age of patient, administration method and the like, and the daily dose for an adult is generally isoleucine 0.5-30.0 g, leucine 1.0-60.0 g, and valine 0.5-30.0 g, preferably isoleucine 2.0-10.0 g, leucine 3.0-20.0 g, and valine 2.0-10.0 g, and more preferably isoleucine 2.5-3.5 g, leucine 5.0-7.0 g, and valine 3.0-4.0 g. When component (1) is composed of three kinds of branched-chain amino acids of isoleucine, leucine and valine, the total daily dose of the three kinds of branched-chain amino acids for an adult is generally 2.0-50.0 g, preferably 3.0-30.0 g. This is administered generally in 1 to 6 portions, preferably 1 to 3 portions, per day as necessary.

While the dose of component (2) varies depending on the pathology and age of patient, administration method and the like, a weekly dose for an adult is generally 500-2000 mg/m², preferably 750-1350 mg/m².

The dose and administration frequency of component (3) can be respectively determine for each medicament according to the pathology and age of patient, administration method and the like. For example, 200-500 mg/m² of 5-fluorouracil is preferable for one cycle for an adult. When tegafur or tegafur-gimeracil-oteracil potassium is used, a tegafur equivalent of 40-60 mg/administration is preferable for an adult.

In the therapeutic drug for pancreatic cancer and/or biliary tract cancer of the present invention, components (1) and (2) as separate preparations may be administered in the same or different administration form(s), or components (1) and (2) may be contained in one kind of preparation.

When the therapeutic drug for pancreatic cancer and/or biliary tract cancer of the present invention further contains component (3), components (1)-(3) may be administered as separate preparations or in a combination of a preparation containing any two kinds thereof and a preparation containing the remaining one kind in the same or different administration form(s), or all of components (1)-(3) may be contained in one kind of preparation.

When components (1) and (2) are separate preparations, the timing of administrations thereof may be the same or different.

When the therapeutic drug for pancreatic cancer and/or biliary tract cancer of the present invention further contains component (3) wherein components (1)-(3) are separate preparations or in a combination of a preparation containing any two kinds thereof and a preparation containing the remaining one kind, the timing of administrations thereof may also be the same or different.

When the dose of the branched-chain amino acid to be used as component (1) in the therapeutic drug for pancreatic cancer and/or biliary tract cancer of the present invention is to be calculated and branched-chain amino acid has already been ingested or administered for an object different from that of the present invention, for example, the need of general eating habits or treatment of other diseases, it is not necessary to include such amount in the dose calculation. For example, the amount of branched-chain amino acid ingested per day in general eating habits does not need to be deducted in the calculation of the aforementioned daily dose of component (1) in the present invention.

The therapeutic drug for pancreatic cancer and/or biliary tract cancer of the present invention is also particularly useful as a therapeutic drug for advanced pancreatic cancer. In the present specification, the advanced pancreatic cancer refers to pancreatic cancer showing progressed pathology, more specifically, pancreatic cancer showing progressed local extent and lymph node metastasis. For example, it corresponds to stage 3, stage 4a, stage 4b in the General Rules for the Study of Pancreatic Cancer by the Japan Pancreas Society, and stage 2A, stage 2B, stage 3, stage 4 in the international TNM classification. Among these, the present therapeutic drug is particularly useful for advanced pancreatic cancer with distant metastasis to distant lymph node and the like, or pancreatic cancer in stage 4b (General Rules for the Study of Pancreatic Cancer) or stage 4 (TNM classification).

The present invention also provides an enhancer of an anticancer activity of gemcitabine or a salt thereof against pancreatic cancer and/or biliary tract cancer (including bile duct cancer, gall bladder cancer, papillary cancer) (hereinafter to be also simply referred to as “the anticancer activity enhancer of the present invention”).

The anticancer activity enhancer of the present invention contains at least one kind of branched-chain amino acid selected from the group consisting of isoleucine, leucine and valine, and preferably contains three kinds of branched-chain amino acids of isoleucine, leucine and valine.

Isoleucine, leucine, and valine to be contained in the anticancer activity enhancer of the present invention may be similar to those contained in component (1) of the aforementioned therapeutic drug for pancreatic cancer and/or biliary tract cancer of the present invention.

When the anticancer activity enhancer of the present invention contains three kinds of branched-chain amino acids of isoleucine, leucine and valine, the weight ratio of isoleucine, leucine and valine can be determined in the same manner as with the weight ratio of the aforementioned component (1).

The anticancer activity enhancer of the present invention can be formulated as a preparation by mixing at least one kind of branched-chain amino acid selected from the group consisting of isoleucine, leucine and valine, and a pharmacologically acceptable carrier according to a method known per se. The obtained preparation can be administered orally or parenterally (e.g., topical, rectal, intravenous administration etc.). As the “pharmacologically acceptable carrier”, those similar to the carriers usable for the production of the therapeutic drug for pancreatic cancer and/or biliary tract cancer of the present invention can be mentioned. As a specific dosage form, one similar to the dosage form of the therapeutic drug for pancreatic cancer and/or biliary tract cancer of the present invention can be mentioned.

The dose and administration method of the anticancer activity enhancer of the present invention can be determined in the same manner as for the component (1) of the therapeutic drug for pancreatic cancer and/or biliary tract cancer of the present invention.

### EXAMPLES

The present invention is explained in more detail in the following by referring to Experimental Examples, which do not limit the present invention in any manner.

### Experimental Example 1

### Preparation of Medium

### (Medium 1-1)

To the healthy control medium (HCM) described in HEPATOLOGY, vol. 50, No. 6, 2009, 1936-1945 was added 5 wt % fetal bovine serum (FBS) to prepare medium 1-1. A specific preparation method of HCM is as described below.

That is, HCM was prepared by weighing respective amino acids to achieve the composition of Table 1, mixing them, dissolving the mixture in an amino acid-zero medium, and filter sterilization thereof.

TABLE 1
amino acid composition of HCM

| amino acid | composition (nmol/ml) |
|---|---|
| Glycine | 225 |
| L-Alanine | 391 |
| L-Serine | 119 |
| L-Threonine | 142 |
| L-Cystine 2HCl | 38 |
| L-Methionine | 29 |
| L-Glutamine | 564 |
| L-Asparagine | 51 |
| L-Glutamic Acid | 42 |
| L-Aspartic Acid | 3 |
| L-Valine | 249 |
| L-Leucine | 132 |
| L-Isoleucine | 76 |
| L-Phenylalanine | 63 |
| L-Tyrosine | 65 |
| L-Tryptophan | 62 |
| L-Lysine-HCl | 183 |
| L-Arginine-HCl | 78 |
| L-Histidine HCl—H₂O | 83 |
| L-Proline | 204 |

The amino acid-zero medium used for preparing HCM was produced by the following procedures (1)-(6).

(1) dissolving amino acid-free D-MEM (Dulbecco's Modified Eagle Medium) medium (Neutrition free DMEM: Zero medium (5.81 g), 09077-05, 2 each for 500 ml) manufactured by Kyokuto Pharmaceutical Industrial Co., Ltd. in double distilled water (800 ml)
(2) further adding sodium hydrogen carbonate (3.7 g) and glucose (1 g) to be dissolved therein
(3) adjusting to pH 7.4 with HCl
(4) measuring up to 1000 ml with double distilled water
(5) sterile filtration using a 0.22 μm filter
(6) storing at 4° C. until use

### (Medium 1-2)

By an operation similar to that for medium 1-1 except that gemcitabine hydrochloride (0.2 μg/ml) was further added, medium 1-2 was prepared.

### (Medium 1-3)

By an operation similar to that for medium 1-1 except that gemcitabine hydrochloride (0.2 μg/ml) and 5-fluorouracil (0.5 μg/ml) were further added, medium 1-3 was prepared.

### (Medium 2-1)

To the advanced cirrhotic medium (ACM) described in HEPATOLOGY, vol. 50, No. 6, 2009, 1936-1945 was added 10 wt % FBS to prepare medium 2-1. A specific preparation method of ACM is as described below.

That is, ACM was prepared by weighing respective amino acids to achieve the composition of Table 2, mixing them, dissolving the mixture in an amino acid-zero medium, and filter sterilization thereof.

TABLE 2
amino acid composition of ACM

| amino acid | composition (nmol/ml) |
|---|---|
| Glycine | 280 |
| L-Alanine | 307 |
| L-Serine | 151 |
| L-Threonine | 138 |
| L-Cystine 2HCl | 67 |
| L-Methionine | 75 |
| L-Glutamine | 689 |
| L-Asparagine | 64 |
| L-Glutamic Acid | 53 |
| L-Aspartic Acid | 4 |
| L-Valine | 175 |
| L-Leucine | 100 |
| L-Isoleucine | 53 |
| L-Phenylalanine | 99 |
| L-Tyrosine | 133 |
| L-Tryptophan | 45 |
| L-Lysine-HCl | 184 |
| L-Arginine-HCl | 92 |
| L-Histidine HCl—H₂O | 85 |
| L-Proline | 176 |
| Fisher ratio | 1.42 |

The amino acid-zero medium used for preparing ACM was produced by the procedures (1)-(6) mentioned above.

### (Medium 2-2)

By an operation similar to that for medium 2-1 except that gemcitabine hydrochloride (0.2 μg/ml) was further added, medium 2-2 was prepared.

### (Medium 2-3)

By an operation similar to that for medium 2-1 except that gemcitabine hydrochloride (0.2 μg/ml) and 5-fluorouracil (0.5 μg/ml) were further added, medium 2-3 was prepared.

### [Confirmation of Effect of Branched-Chain Amino Acid (BCAA) Addition]

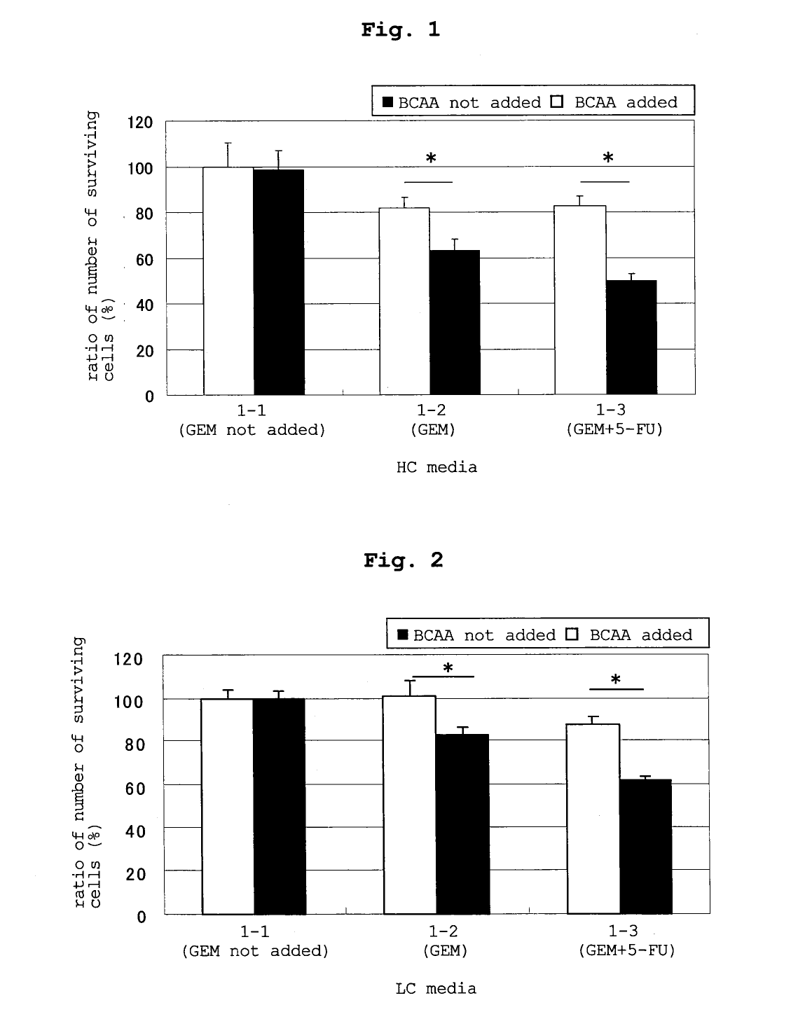

Panc-1 cells, which are cells derived from a human pancreatic cancer cell line, were plated in a 96-well microtiter plate at the cell density of 4000 cells per well, and cultured overnight at 37° C., 5% CO₂ to allow adhesion. The media of the cells were respectively substituted by the above-mentioned media 1-1-1-3 and 2-1-2-3. They were divided into 4 mM BCAA (manufactured by Ajinomoto Co., Inc., trade name “Livact (registered trade mark) granules”, isoleucine:leucine:valine=1:2:1.2 (weight ratio)) addition cells and non-addition cells, and the numbers of surviving Panc-1 cells after culture for 72 hr were compared. The number of surviving cells was counted by ArrayScan: quantitative device for fluorescence microscopy (manufactured by Thermo Fisher Scientific Inc.) after staining the cell nucleus with the Hoechst reagent. The results are shown in FIGS. 1 and 2.

The number of surviving cells (vertical axis) in FIG. 1 is shown in the ratio (%) of the number of respective surviving cells relative to the number of surviving cells when cultured for 72 hr without addition of BCAA to medium 1-1 as 100. Similarly, the number of surviving cells (vertical axis) in FIG. 2 is also shown in the ratio (%) of the number of respective surviving cells relative to the number of surviving cells when cultured for 72 hr without addition of BCAA to medium 2-1 as 100.

As is clear from the results of FIGS. 1 and 2, even when BCAA was added to gemcitabine hydrochloride-free media (media 1-1, 2-1), the number of surviving Panc-1 cells did not change. However, when BCAA was added to the media added with gemcitabine hydrochloride (media 1-2, 2-2), the number of surviving cells significantly decreased. Therefore, it was confirmed that BCAA can enhance the anticancer activity of gemcitabine hydrochloride against pancreatic cancer.

In addition, when BCAA was added to the media added with gemcitabine hydrochloride and 5-fluorouracil (media 1-3, 2-3), the number of surviving cells decreased.

### Experimental Example 2

Human pancreatic cancer cells (panc-1) (2×10⁶ cells/100 μL) were subcutaneously transplanted to BALB/c nude mice (female, 6-week-old). One week after the transplantation, they were grouped based on the tumor diameter into 5 groups, and chemotherapeutic agents were respectively administered according to the following schedule.

groups 1 and 2: intraperitoneal administration of gemcitabine hydrochloride 60 mg/kg twice/week for 3 weeks→cessation of the drug (3 weeks)→intraperitoneal administration of gemcitabine hydrochloride 100 mg/kg twice/week for 3 weeks
groups 3 and 4: intraperitoneal administration of gemcitabine hydrochloride 60 mg/kg and 5-fluorouracil 20 mg/kg twice/week for 3 weeks→cessation of the drug (3 weeks)→intraperitoneal administration of gemcitabine hydrochloride 100 mg/kg and 5-fluorouracil 20 mg/kg twice/week for 3 weeks control group (control): intraperitoneal administration of saline twice/week for 3 weeks→cessation of the drug (3 weeks)→intraperitoneal administration of saline twice/week for 3 weeks

Groups 1, 3 and control group were given a general feed (CRF-1, manufactured by Oriental Yeast Co. Ltd.) throughout the entire period. Groups 2, 4 were given a mixed feed containing 3% BCAA (manufactured by Ajinomoto Co., Inc., trade name “Livact (registered trade mark) granules”, isoleucine:leucine:valine=1:2:1.2 (weight ratio)) during the chemotherapeutic agent (gemcitabine hydrochloride, 5-fluorouracil) dosing period, and the feed was changed to a general feed (CRF-1) during cessation of the drug.

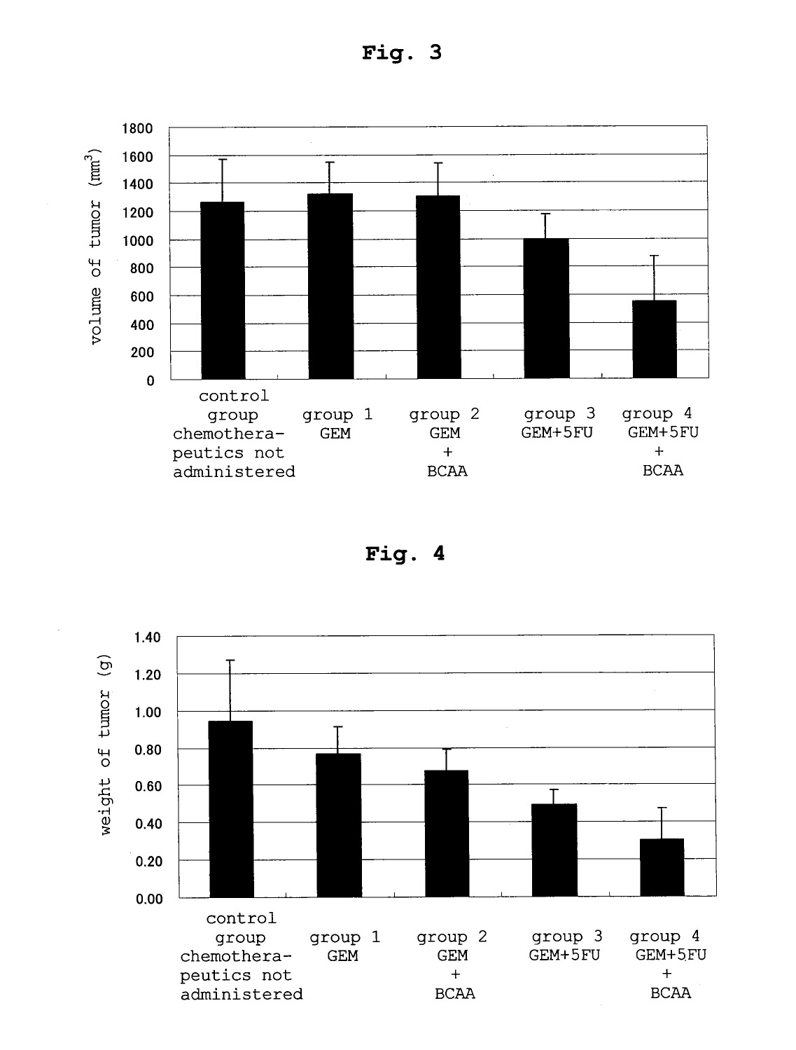

The mice were autopsied at 76 days after the transplantation, the tumor was isolated, and the volume and weight were measured. The measurement results of tumor volume are shown in FIG. 3, and the measurement results of tumor weight are shown in FIG. 4.

As is clear from the results of FIGS. 3 and 4, the tumor weight decreased by using BCAA in combination with gemcitabine hydrochloride. When BCAA was used in combination with gemcitabine hydrochloride and 5-fluorouracil, the volume and weight of the tumor decreased.

### Experimental Example 3

“Livact (registered trade mark) granules” (BCAA content of one dosage: L-isoleucine 952 mg, L-leucine 1904 mg, L-valine 1144 mg) 4.15 g/dosage was administered for 3 months at 3 dosages/day to one case of stage 4b pancreatic cancer patient (male, 70's) with distant metastasis to distant lymph node and the like, who was prescribed with “Gemzar (registered trade mark)” (dose: continuous administration at 1000 mg/body/week as gemcitabine for 2 weeks, followed by cessation of the drug for 1 week is one cycle, and the cycle is repeated for 3 months), and a combination preparation “TS-1 (registered trade mark)” containing tegafur (dose: continuous administration at 100 mg/body/day of tegafur equivalent for 2 weeks, followed by cessation of the drug for 1 week is one cycle, and the cycle is repeated for 3 months). As a result, the maximum tumor diameter as measured by CT image, which was 47 mm before administration of the Livact granules, decreased to 35 mm after Livact granules administration.

On the other hand, the maximum tumor diameter of 4 cases of the same stage 4b pancreatic cancer patients with tumor having a diameter of 35 mm-54 mm (39.9 mm, 35.1 mm, 40.5 mm, 37.1 mm), who were prescribed with “Gemzar” (dose: continuous administration at 1000 mg/body/week as gemcitabine for 2 weeks, followed by cessation of the drug for 1 week is one cycle, and the cycle is repeated for 3 months), and “TS-1” (dose: continuous administration at 100 mg/body/day of tegafur equivalent for 2 weeks, followed by cessation of the drug for 1 week is one cycle, and the cycle is repeated for 3 months), but not prescribed with “Livact granules”, was measured by CT image, and the average thereof was calculated. As a result, the maximum tumor diameter before administration of Gemzar and TS-1 was 38.2±1.26 mm, and 47.9±6.91 mm 3 months after the administration.

### INDUSTRIAL APPLICABILITY

According to the present invention, a highly effective therapeutic drug for pancreatic cancer and/or biliary tract cancer can be provided. According to the present invention, moreover, an enhancer of an anticancer activity of gemcitabine or a salt thereof against pancreatic cancer and/or biliary tract cancer can be provided.

Obviously, numerous modifications and variations of the present invention are possible in light of the above teachings. It is therefore to be understood that, within the scope of the appended claims, the invention may be practiced otherwise than as specifically described herein.

All patents and other references mentioned above are incorporated in full herein by this reference, the same as if set forth at length.

1. A therapeutic drug, comprising:
(1) at least one kind of branched-chain amino acid selected from the group consisting of isoleucine, leucine, and valine; and
(2) gemcitabine or a salt thereof.

2. A therapeutic drug according to claim 1, further comprising:
(3) at least one compound selected from the group consisting of a 5-fluorouracil compound, a platinum compound, a taxane compound, a vinca alkaloid compound, an anticancer tyrosine kinase inhibitory compound, and an anticancer monoclonal antibody.

3. A therapeutic drug according to claim 1, comprising a first preparation which comprises said at least one kind of branched-chain amino acid (1) in combination with a second preparation which comprises said gemcitabine or a salt thereof (2).

4. A therapeutic drug according to claim 3, further comprising a third preparation comprising said at least one compound selected from the group consisting of a 5-fluorouracil compound, a platinum compound, a taxane compound, a vinca alkaloid compound, an anticancer tyrosine kinase inhibitory compound, and an anticancer monoclonal antibody (3) in combination with said first preparation and said second preparation.

5. A therapeutic drug according to claim 2, which comprises a 5-fluorouracil compound.

6. A therapeutic drug according to claim 5, which comprises a 5-fluorouracil compound selected from the group consisting of 5-fluorouracil, tegafur, tegafur-gimeracil-oteracil potassium, and capecitabine.

7. A therapeutic drug according to claim 1, which comprises isoleucine, leucine, and valine.

8. A therapeutic drug according to claim 7, wherein said isoleucine, leucine, and valine are present in a weight ratio of 1:1-3:0.5-2.0.

9. A therapeutic drug according to claim 7, wherein said isoleucine, leucine, and valine are present in a weight ratio of 1:1.5-2.5:0.8-1.7.

10. A therapeutic drug according to claim 7, wherein said isoleucine, leucine, and valine are present in a weight ratio of 1:1.9-2.2:1.1-1.3.

11. A therapeutic drug according to claim 1, which comprises gemcitabine hydrochloride.

12. A therapeutic drug according to claim 2, which comprises (1) isoleucine, leucine, and valine, (2) gemcitabine hydrochloride and (3) a 5-fluorouracil compound.

13. A therapeutic drug according to claim 4, comprising the first preparation which comprises isoleucine, leucine, and valine in combination with the second preparation which comprises gemcitabine hydrochloride and the third preparation which comprises a 5-fluorouracil compound.

14. A therapeutic drug according to claim 2, comprising a first preparation which comprises isoleucine, leucine and valine, and a 5-fluorouracil compound in combination with a second preparation which comprises gemcitabine hydrochloride.

15. A method for enhancing an anticancer activity of gemcitabine or a salt thereof against pancreatic cancer and/or biliary tract cancer, comprising administering to a subject in need thereof an effective amount of at least one kind of branched-chain amino acid selected from the group consisting of isoleucine, leucine, and valine.

16. A method according to claim 15, comprising administering isoleucine, leucine, and valine.

17. A method of treating pancreatic cancer and/or biliary tract cancer, comprising administering to a subject in need thereof an effective amount of:
(1) at least one kind of branched-chain amino acid selected from the group consisting of isoleucine, leucine, and valine; and
(2) gemcitabine or a salt thereof.

18. A method according to claim 17, further comprising administering an effective amount of:
(3) at least one compound selected from the group consisting of a 5-fluorouracil compound, a platinum compound, a taxane compound, a vinca alkaloid compound, an anticancer tyrosine kinase inhibitory compound, and an anticancer monoclonal antibody.

19. A method according to claim 17, wherein said pancreatic cancer and/or biliary tract cancer is advanced pancreatic cancer.

20. A method according to claim 17, which comprises administering a 5-fluorouracil compound.

21. A method according to claim 17, which comprises administering a 5-fluorouracil compound selected from the group consisting of 5-fluorouracil, tegafur, tegafur-gimeracil-oteracil potassium, and capecitabine.

22. A method according to claim 17, which comprises administering isoleucine, leucine, and valine.

23. A method according to claim 17, wherein said isoleucine, leucine, and valine are administered in a weight ratio of 1:1-3:0.5-2.0.

24. A method according to claim 17, which comprises administering (1) isoleucine, leucine, and valine, (2) gemcitabine hydrochloride and (3) a 5-fluorouracil compound.
